(19) United States
(12) Patent Application Publication
(10) Pub. No.: US 2008/0312571 A1
(43) Pub. Date: Dec. 18, 2008

(54) Foot Support

(75) Inventor: John Charles Waller, New South Wales (AU)

Correspondence Address: CARTER, DELUCA, FARRELL & SCHMIDT, LLP, 445 BROAD HOLLOW ROAD, SUITE 420, MELVILLE, NY, 11747, US

(73) Assignee: WESTON TRADERS PTY LIMITED, AN AUSTRALIAN COMPANY, Weston, New South Wales (AU)

(21) Appl. No.: 12/093,860

(22) PCT Filed: Nov. 22, 2006
(86) PCT No.: PCT/AU2006/001766

§ 371 Date: Aug. 7, 2008

(30) Foreign Application Priority Data
Nov. 22, 2005 (AU) 2005906491

(51) Int. Cl. A61F 5/00

(52) U.S. Cl. 602/27

(57) ABSTRACT

A foot support (1) including a first length (15) of flexible material to extend about a user's leg (5) adjacent an ankle (12) of the leg (5) to secure the support to the leg (5). A second length (20) of flexible material being attached to the first length (15) so as to extend therefrom to form a loop to pass under a foot (10) below the ankle (12). A third length (27) of flexible material attached to the first length (15) to extend therefrom to be attached to a shoe (29) worn on the leg (5) at a position forward of the ankle (12) so that in use the third length (27) extends at an acute angle downward from the leg (5) to the shoe (29).

### FIELD OF THE INVENTION

The present invention relates to limb supports and, in particular but not exclusively to a foot support for sufferer's of “Foot Drop” or the like.

### BACKGROUND OF THE INVENTION

Limbs of the body throughout their life can suffer a number of debilitating symptoms reducing the control of that limb by a user.

“Foot Drop” is such a symptom whereby dropping of the front of the foot due to a weakness or paralysis of the anterior muscles of the lower leg results in a steppage gait in which the advancing foot is left higher so that the toes may clear the ground. Foot Drop can be due to a number of conditions including injury to the muscles that dorsiflex the foot or to the nerves of those muscles, neuropathy, multiple sclerosis, stroke, drug toxicity, or diabetes.

The most common method of treating “Foot Drop” is to provide an ankle foot orthosis. That is, a brace (usually plastic) worn on the lower leg and foot to support the ankle, hold the foot and ankle in the correct position, and correct “Foot Drop”.

Such braces however have been found not to provide sufficient mobility of the ankle, foot and lower leg as the brace includes a fixed method of holding the foot at a right angle to the lower leg which causes various levels of muscle wastage in the lower leg and foot through inactivity. Further, foot braces cause jarring when the patient walks up or down inclines damaging the hip and knee. The braces that are hinged only allow approximately 20 degrees of forward movement.

### OBJECT OF THE INVENTION

It is an object of the present invention to overcome or ameliorate some of the disadvantages of the prior art, or at least to provide a useful alternative.

### SUMMARY OF THE INVENTION

There is firstly disclosed herein a foot support including:

a first length of flexible material to extend about a user's leg adjacent an ankle of the leg to secure the support to the leg;

a second length of flexible material being attached to the first length so as to extend therefrom to form a loop to pass under a foot below the ankle;

a third length of flexible material attached to the first length to extend therefrom to be attached to a shoe worn on the leg at a position forward of the ankle so that in use the third length extends at an acute angle downward from the leg to the shoe.

Preferably, in an expanded configuration said second length is attached to the first length adjacent a respective end edge to extend longitudinally away from, and perpendicular to, said first length.

Preferably, including fastening means operable to secure together respective free ends of said first and second lengths.

Preferably, said fastening means includes hook and loop type fasteners.

Preferably, said support inhibits said shoe and thereby said foot moving beyond a perpendicular to said leg.

Preferably, including adjustment means to alter the length of said third length.

Preferably, said third length engages the shoe at laces.

Preferably, said lengths are strips of synthetic material.

Preferably, free ends of said lengths include attachment means.

Preferably, said attachment means include hooks.

Preferably, said attachment means include rings.

### BRIEF DESCRIPTION OF THE DRAWINGS

A preferred embodiment of the invention will now be described, by way of example only, with reference to the accompanying drawings, wherein:

FIG. 1 is a plan view of an embodiment of the present invention in an expanded configuration;

FIGS. 2 to 9 are photographic representations of the foot support of FIG. 1 being positioned around the ankle of a user; and

FIGS. 10A, 10B are hook attachments for use with the support of FIG. 1;

### DETAILED DESCRIPTION OF THE PREFERRED EMBODIMENTS

In the accompanying drawings, there is schematically depicted herein a foot support 1 to be removably fastened to a leg 5 of a user to maintain the foot 10 substantially perpendicular to the leg 5 to overcome or at least inhibit “Foot Drop”. The support 1 includes a first length 15 of flexible material to extend about a user's leg 5 adjacent an ankle 12 of the leg 5 to secure the support 1 to the leg 5. A second length 20 of flexible material attached to the first length 15 so as to extend therefrom to form a loop to pass under a foot 5 below the ankle 12. A third length 27 of flexible material attached to the first length 15 to extend therefrom to be attached to a shoe 29 worn on the leg 5 at a position forward of the ankle 12 so that in use the third length 27 extends at an acute angle downward from the leg S to the shoe 29. The first and second lengths 15, 20 each have a pair of longitudinally extending side edges 25, 30 and a pair of end edges 35, 40.

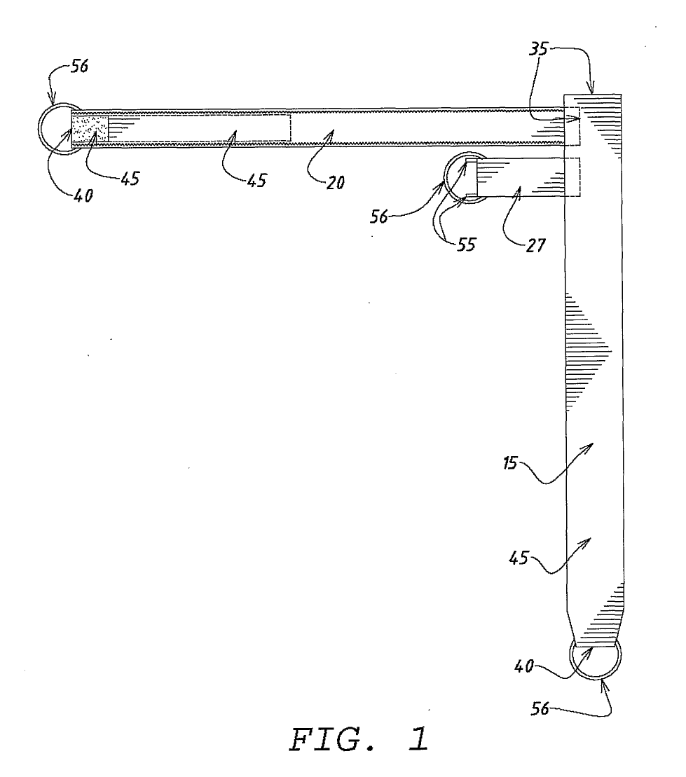

As best seen in FIG. 1, the second length 20 is attached to the first length 15 adjacent respective end edges 35 to extend longitudinally away from, and perpendicular to, the first length 15. The support 1 in an expanded configuration forms a substantially “L” shape. The first and second lengths 15, 20 include fastening means 45 which are adapted to secure the lengths 15, 20 to preferably the middle of the first length 15. Fastening means 45 in the preferred form would include hook and loop type fasteners. However, any typical fastening means 45 could be utilised. In FIG. 1, the fastening means 45 forms the top surface of length 15. In the second length 20 the fastening means 45 overlaps the top and bottom surfaces as shown. The first length 15 is locatable above the ankle 12 of the user to secure the support 1 to the leg 5 in a substantially fixed position. The first length 15 is secured to itself by another fastening means 45. The second length 20 is operable to extend from one side of the leg 5 about a heel 50 of the user to connect to the first length 15 at the opposite side of the leg 5. The second length 20 is secured to the first length 15 by the fastening means 45. The support 1 inhibits the shoe 29 and thereby the foot 10 moving beyond a perpendicular to the leg 5. The third length 27 could also include adjustment means or the like to alter the length between the first length 15 and shoe 29 to maintain the third length 27 in tension.

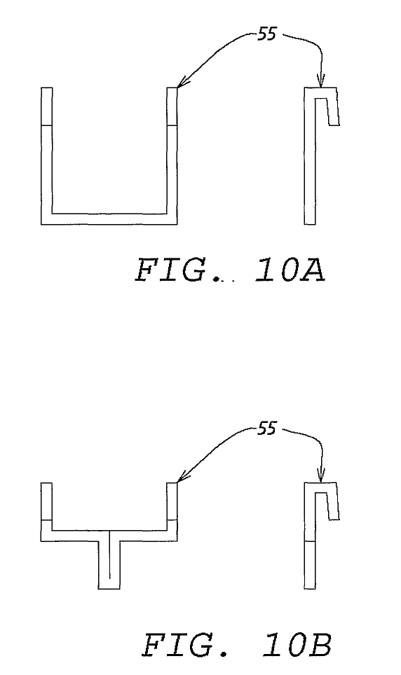

In the preferred form, the length 27 would be elasticized and can be attached to any part of a shoe 29 by way of laces. As shown in FIGS. 10A, 10B hook 55 could be utilised at the end of length 27 to assist in attaching the support 1 to the shoe 29. Clips or other such attachment devices could also be utilised. The lengths 15, 20, 27 can also include loops or rings 56 to assist the user to correctly position the support 1 on the leg 5.

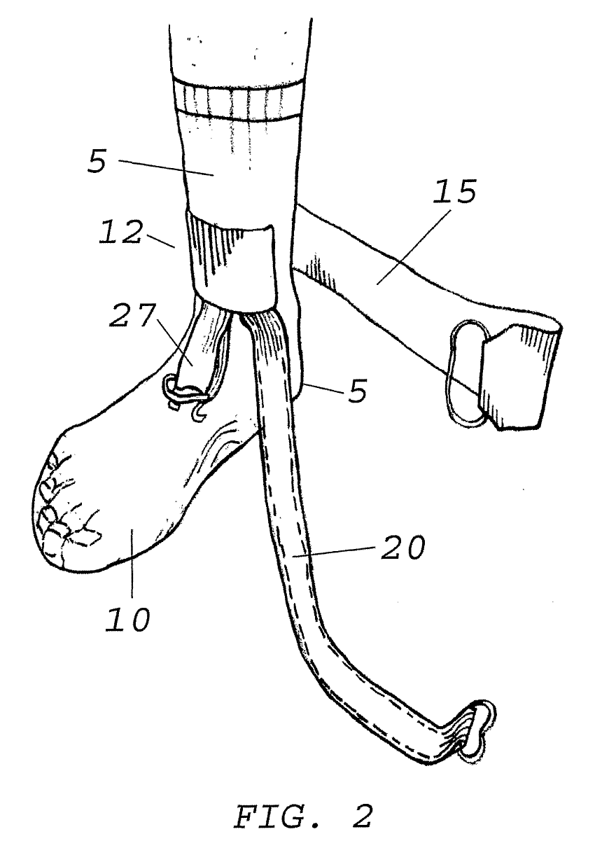

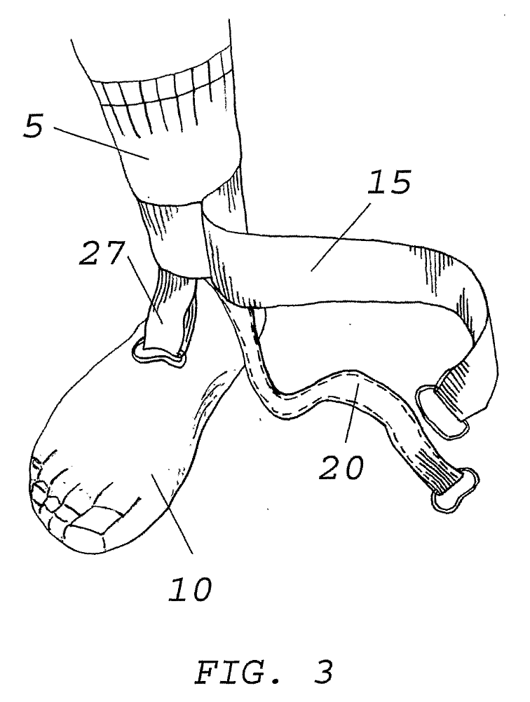

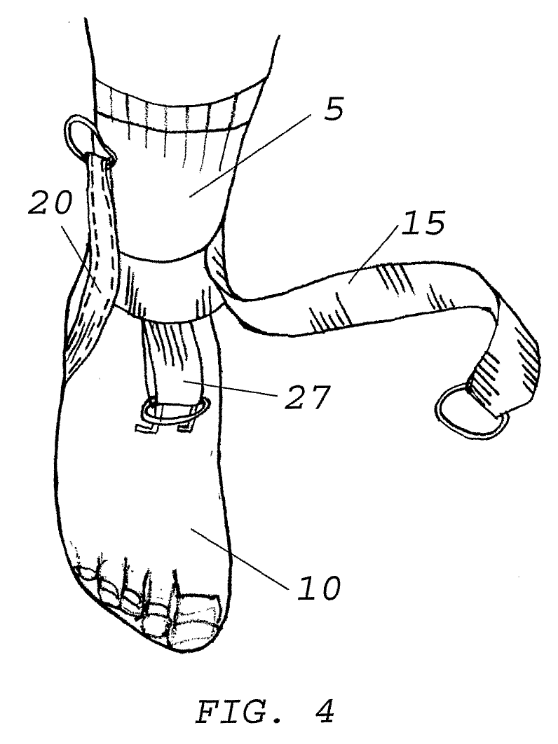

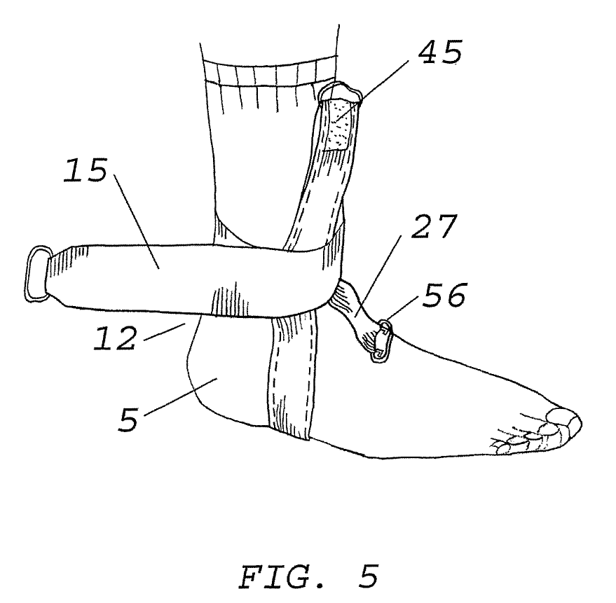

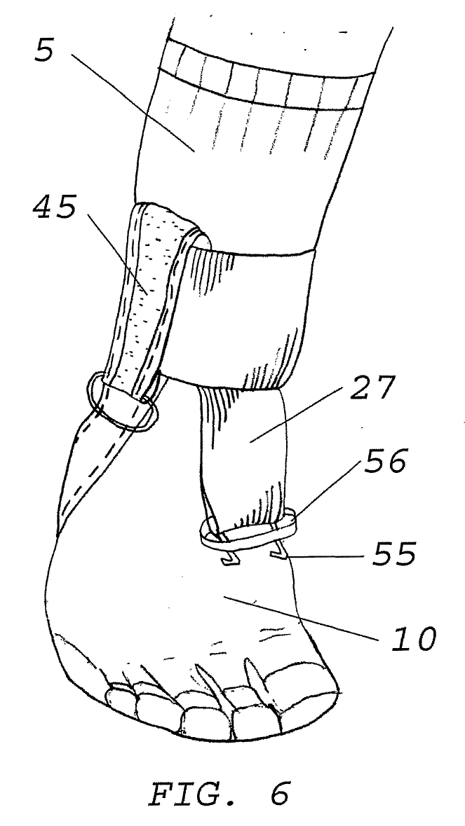

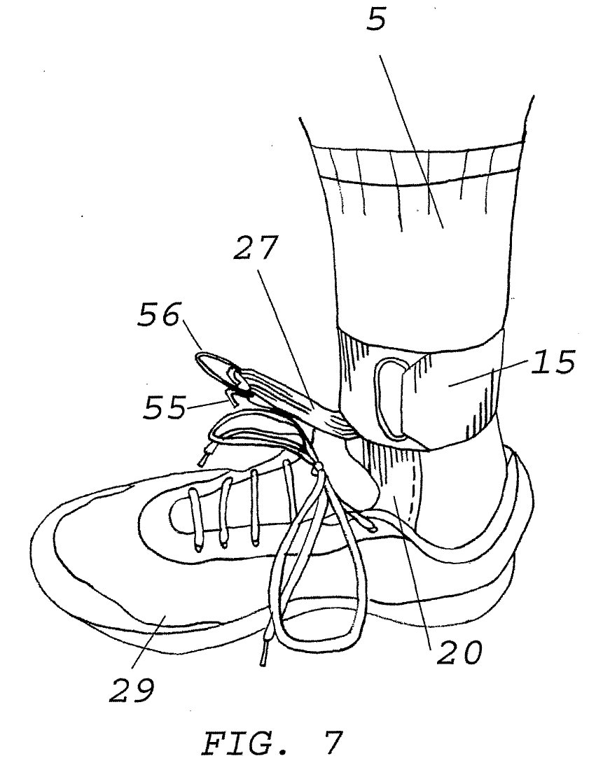

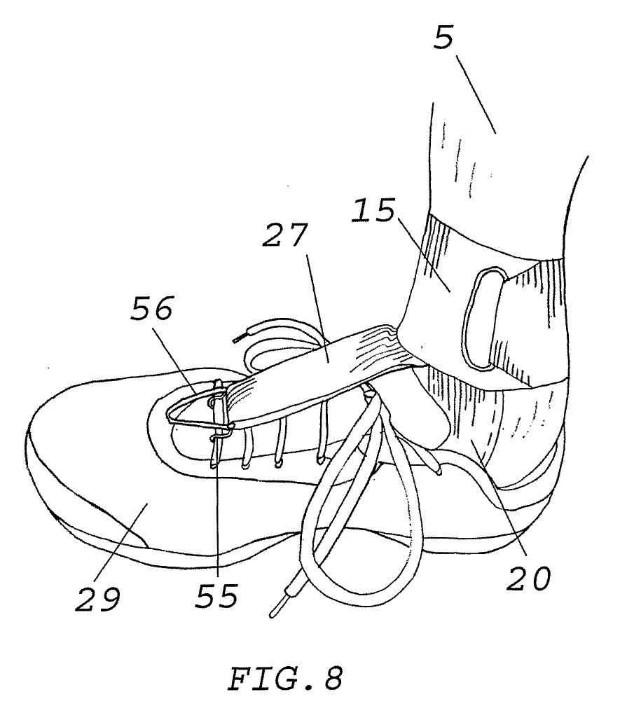

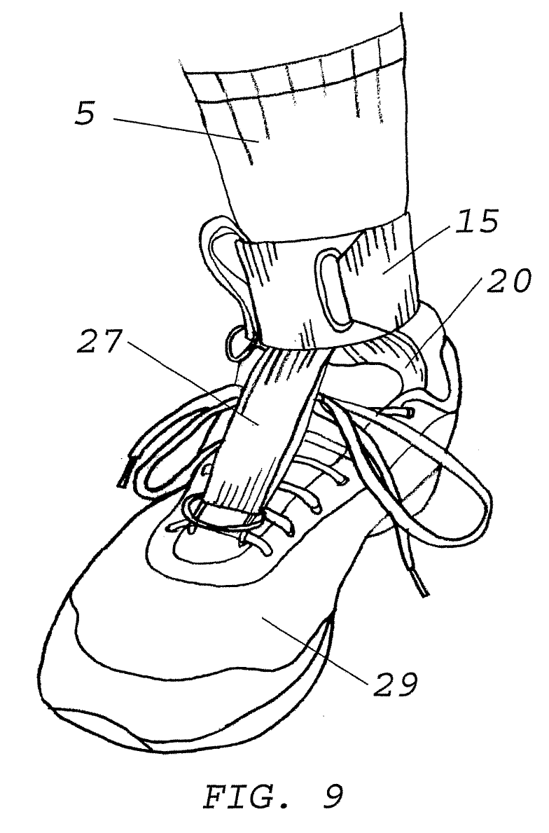

In use, and as set out in FIGS. 2 to 9, a sufferer of “Foot Drop” or the like would wrap the first length 15 at a point above the ankle 12 of the leg 5 connecting the respective fastening means 45 together to secure the support 1 in a substantially fixed position to the leg 5. The second length 20 would then be wrapped around the ankle 12 from one side of the leg 5 to the opposite side securing the free ends 40 by way of the fastening means 45 back to the first length 15. The user would then place their foot 10 in a shoe 29 as normal and once secured in position the third length 27 is attached from the first length 15 to the laces of the shoe 29. The third length 27 is tensioned so the foot 10 is substantially perpendicular to the leg 5.

Advantageously, the present invention at least in the preferred embodiment provides a method of raising the front of a shoe 29 to counteract the effect of “Foot Drop” caused by various medical conditions. Also provided is full mobility of the ankle 12, foot 10 and lower leg 5 as close to normal use of the foot is possible with the present invention. Accordingly, the muscles of the leg 5 and foot 10 retain their shape and use and this is important for people who are being rehabilitated from illnesses, such as, heart, stroke and neuropathies. Further, a person suffering from these conditions can maintain as normal a lifestyle as possible without the inconvenience of having to wear a fixed brace. Also, the support 1 reduces the jarring caused by a foot brace when walking up or down inclines, and reduces damage to the hip and knee. The support 1 further provides smoother step movement when walking up/down steps and up/down inclines and creates a safer platform when traversing on uneven ground. Further, due to the support's size and non-intrusiveness it allows the user to wear normal apparel and any type of shoe 29. Further, there are no modifications required to existing shoes 29.

Although the invention has been described with reference to specific examples, it will be appreciated by those skilled in the art that the invention may be embodied in many other forms.

1. A foot support comprising:
a first length of flexible material to extend about a user's leg adjacent an ankle of the leg to secure the support to the leg;
a second length of flexible material being attached to the first length so as to extend therefrom to form a loop to pass under a foot below the ankle;
a third length of flexible material attached to the first length to extend therefrom to be attached to a shoe worn on the leg at a position forward of the ankle so that in use the third length extends at an acute angle downward from the leg to the shoe.

2. The foot support of claim 1, whereby in an expanded configuration said second length is attached to the first length adjacent a respective end edge to extend longitudinally away from, and perpendicular to, said first length.

3. The foot support of claim 1, further comprising fastening means operable to secure together respective free ends of said first and second lengths.

4. The foot support of claim 3, wherein said fastening means includes hook and loop type fasteners.

5. The foot support of claim 1, whereby said support inhibits said shoe and thereby said foot moving beyond a perpendicular to said leg.

6. The foot support of claim 1, further comprising adjustment means to alter the length of said third length.

7. The foot support of claim 6, wherein said third length engages the shoe at laces.

8. The foot support of claim 1, wherein said lengths are strips of synthetic material.

9. The foot support of claim 1, wherein free ends of said lengths include attachment means.

10. The foot support of claim 9, wherein said attachment means include hooks.

11. The foot support of claim 9, wherein said attachment means include rings.
